(19) United States
(12) Patent Application Publication
(10) Pub. No.: US 2007/0189971 A1
(43) Pub. Date: Aug. 16, 2007

(54) Contrast Agents

(76) Inventor: Hanno Priebe, Nydalen (NO)

Correspondence Address: GE Healthcare, Inc;IP Department, 101 Carnegie Center, Princeton, NJ, 08540, US

(21) Appl. No.: 11/674,204

(22) Filed: Feb. 13, 2007

(30) Foreign Application Priority Data
Feb. 14, 2006 (NO) 20060711

(51) Int. Cl. A61K 49/04

(52) U.S. Cl. 424/9.44; 544/221

(57) ABSTRACT

The present invention relates to a class of compounds and to diagnostic compositions containing such compounds where the compounds are iodine containing compounds. More specifically the iodine containing compounds are chemical compounds containing a cyanuric acid scaffolding moiety allowing for the arrangement of three iodinated phenyl groups bound thereto.

The invention also relates to the use of such diagnostic compositions as contrast agents in diagnostic imaging and in particular in X-ray imaging and to contrast media containing such compounds.

### TECHNICAL FIELD OF THE INVENTION

The present invention relates to a class of compounds and to diagnostic compositions containing such compounds where the compounds are iodine containing compounds. More specifically the iodine containing compounds are chemical compounds containing a cyanuric acid scaffolding moiety allowing for the arrangement of three iodinated phenyl groups bound thereto.

The invention also relates to the use of such diagnostic compositions as contrast agents in diagnostic imaging and in particular in X-ray imaging and to contrast media containing such compounds.

### Description of Related art

All diagnostic imaging is based on the achievement of different signal levels from different structures within the body. Thus in X-ray imaging for example, for a given body structure to be visible in the image, the X-ray attenuation by that structure must differ from that of the surrounding tissues. The difference in signal between the body structure and its surroundings is frequently termed contrast and much effort has been devoted to means of enhancing contrast in diagnostic imaging since the greater the contrast between a body structure and its surroundings the higher the quality of the images and the greater their value to the physician performing the diagnosis. Moreover, the greater the contrast the smaller the body structures that may be visualized in the imaging procedures, i.e. increased contrast can lead to increased spatial resolution.

The diagnostic quality of images is strongly dependent on the inherent noise level in the imaging procedure, and the ratio of the contrast level to the noise level can thus be seen to represent an effective diagnostic quality factor for diagnostic images.

Achieving improvement in such a diagnostic quality factor has long been and still remains an important goal. In techniques such as X-ray, magnetic resonance imaging (MRI) and ultrasound, one approach to improving the diagnostic quality factor has been to introduce contrast enhancing materials formulated as contrast media into the body region being imaged.

Thus in X-ray early examples of contrast agents were insoluble inorganic barium salts which enhanced X-ray attenuation in the body zones into which they distributed. For the last 50 years the field of X-ray contrast agents has been dominated by soluble iodine containing compounds. Commercial available contrast media containing iodinated contrast agents are usually classified as ionic monomers such as diatrizoate (marketed e.g. under the trade name Gastrografen™), ionic dimers such as ioxaglate (marketed e.g. under the trade name Hexabrix™), nonionic monomers such as iohexol (marketed e.g. under the trade name Omnipaque™), iopamidol (marketed e.g. under the trade name Isovue™), iomeprol (marketed e.g. under the trade name lomeron™) and the non-ionic dimer iodixanol (marketed under the trade name and Visipaque™).

The most widely used commercial non-ionic X-ray contrast agents such as those mentioned above are considered safe. Contrast media containing iodinated contrast agents are used in more that 20 millions of X-ray examinations annually in the USA and the number of adverse reactions is considered acceptable. However, since a contrast enhanced X-ray examination will require up to about 200 ml contrast media administered in a total dose, there is a continuous drive to provide improved contrast media.

The utility of the contrast media is governed largely by its toxicity, by its diagnostic efficacy, by adverse effects it may have on the subject to which the contrast medium is administered and by the ease of storage and ease of administration. Since such media are conventionally used for diagnostic purposes rather than to achieve direct therapeutic effect, it is generally desirable to provide media having as little as possible effect on the various biological mechanisms of the cells or the body as this will lead to lower toxicity and lower adverse clinical effect. The toxicity and adverse biological effects of a contrast medium are contributed to by the components of the formulation medium, e.g. the solvent or carrier as well as the contrast agent itself and its components such as ions for the ionic contrast agents and also by its metabolites.

The major contributing factors to the toxicity of the contrast medium are identified as the chemotoxicity of the contrast agent, the osmolality of the contrast medium and the ionic composition or lack thereof of the contrast medium.

Desirable characteristics of an iodinated contrast agent are low toxicity of the compound itself (chemotoxicity), low viscosity of the contrast medium wherein the compound is dissolved, low osmolality of the contrast medium and a high iodine content (frequently measured in g iodine per ml of the formulated contrast medium for administration). The iodinated contrast agent must also be completely soluble in the formulation medium, usually an aqueous medium, and remain in solution during storage.

The osmolality of the commercial products, and in particular of the non-ionic compounds is acceptable for most media containing dimers and non-ionic monomers although there is still room for improvement. In coronary angiography for example, injection into the circulatory system of a bolus dose of contrast medium has caused severe side effects. In this procedure contrast medium rather than blood flows through the system for a short period of time, and differences in the chemical and physiochemical nature of the contrast medium and the blood that it replaces can cause undesirable adverse effects such as arrhythmias, QT prolongation and reduction in cardiac contractive force. Such effects are seen in particular with ionic contrast agents where osmotoxic effects are associated with hypertonicity of the injected contrast medium. Contrast media that are isotonic or slightly hypotonic with the body fluids are particularly desired. Low osmolar contrast media have low renal toxicity which is particularly desirable. The osmolality is a function of the number of particles per volume unit of the formulated contrast medium.

To keep the injection volume of the contrast media as low as possible it is highly desirable to formulate contrast media with high concentration of iodine/ml, and still maintain the osmolality of the media at a low level, preferably below or close to isotonicity. The development of non-ionic monomeric contrast agents and in particular non-ionic bis(triiodophenyl) dimers such as iodixanol (EP patent 108638) has provided contrast media with reduced osmotoxicity allowing contrast effective iodine concentration to be achieved with hypotonic solution, and has even allowed correction of ionic imbalance by inclusion of plasma ions while still maintaining the contrast medium Visipaque™ at the desired osmolality (WO 90/01194 and WO 91/13636).

The X-ray contrast media at commercial high iodine concentration have relative high viscosity, ranging from about 15 to about 60 mPas at ambient temperature. Generally, contrast media where the contrast enhancing agent is a dimer has higher viscosity than the corresponding contrast media where the contrast enhancing agent is the monomer corresponding to the dimer. Such high viscosities may pose problems to the administrators of the contrast medium, requiring relatively large bore needles or high applied pressure, and are particularly pronounced in pediatric radiography and in radiographic techniques which require rapid bolus administration, e.g. in angiography.

X-ray contrast agents of high molecular weight has been proposed, e.g. polymers with substituted triiodinated phenyl groups grafted on the polymer, see EP 354836, EP 436316 and U.S. Pat. No. 5,019,370. Further, WO 9501966, EP 782563 and U.S. Pat. No. 5,817,873 read on compounds having e.g. 3 and 4 substituted triiodinated phenyl groups arranged linearly or around a central core. However, none of these proposed compounds are on the market.

Hence there still exists a desire to develop contrast agents that solves one or more of the problems discussed. Such agents should ideally have improved properties over the soluble iodine containing compounds in one or more of the following properties: renal toxicity, osmolality, viscosity, solubility, injection volumes/iodine concentration and attenuation/radiation dose.

### SUMMARY OF THE INVENTION

The present invention provides compounds useful in contrast media having improved properties over the known media with regards to at least one of the following criteria: osmolality (and hence the renal toxicity), viscosity, iodine concentration and solubility. The contrast media comprises iodine containing contrast enhancing compounds where iodine containing compounds are chemical compounds containing a scaffolding moiety allowing for the arrangement of three iodinated phenyl groups bound to thereto. The iodine containing contrast enhancing compounds can be synthesized from commercially available and relatively inexpensive starting materials.

### DETAILED DESCRIPTION OF THE INVENTION

The contrast enhancing compounds are synthetic chemical compounds of formula (I)

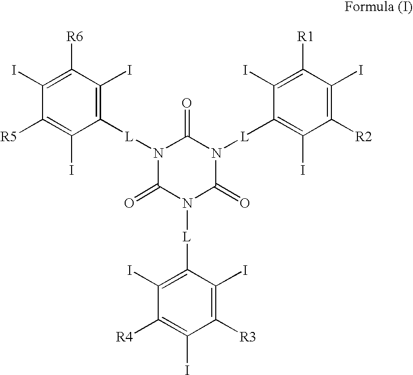

In formula (I) each L are the same or different and denote a linker of formula —N(Ac)—CH₂—CHOH—CH₂— wherein Ac denotes a C₁ to C₅ organic acid residue such as the formyl, acetyl, propionyl, butyryl, isobutyryl and valeriyl. Hydroxylated and metoxylated organic acid moieties are also preferred. Particularly preferred are the acetyl, hydroxyacetyl and metoxyacetyl moieties.

Each of the substituents R¹, R², R³, R⁴, R⁵ and R⁶ (hereinafter collectively denoted R group(s)) may be the same or different and denote a hydrogen atom or a non-ionic hydrophilic moiety, provided that at least one R group is a hydrophilic moiety or salts or optical active isomers thereof.

The non-ionic hydrophilic moieties may be any of the non-ionizing groups conventionally used to enhance water solubility. Suitable groups include straight chain or branched chain C₁₋₁₀ alkyl groups, preferably C₁₋₅ alkyl groups, optionally with one or more CH₂ or CH moieties replaced by oxygen or nitrogen atoms and optionally substituted by one or more groups selected from oxo, hydroxyl, amino or carboxyl derivative, and oxo substituted sulphur and phosphorus atoms. Particular examples include polyhydroxyalkyl, hydroxyalkoxyalkyl and hydroxypolyalkoxyalkyl and such groups attached to the phenyl group via an amide linkage such as hydroxyalkylaminocarbonyl, N-alkyl-hydroxyalkylaminocarbonyl and bis-hydroxyalkylaminocarbonyl groups.

In a preferred embodiment the hydrophilic moieties contain 1 to 6 hydroxy groups, preferably 1 to 3 hydroxy groups.

The R groups of the formulas listed below are preferred:
—CONH—CH₂—CH₂—OH
—CONH—CH₂—CHOH—CH₂—OH
—CON(CH₃)CH₂—CHOH—CH₂OH
—CONH—CH—(CH₂—OH)₂
—CON—(CH₂—CH₂—OH)₂
—CONH₂
—CONHCH₃
—NHCOCH₂OH
—N(COCH₃)H
—N(COCH₃) C₁₋₃ alkyl
—N(COCH₃)-mono, bis or tris-hydroxy C₁₋₄ alkyl
—N(COCH₂OH)-hydrogen, mono, bis or tris-hydroxy C₁₋₄ alkyl
—N(CO—CHOH—CH₂OH)-hydrogen, mono, bis or trihydroxylated C₁₋₄ alkyl.
N(CO—CHOH—CHOH—CH₂OH)-hydrogen, mono, bis or trihydroxylated C₁₋₄ alkyl.
—N(COCH₂OH)₂
—CON(CH₂—CHOH—CH₂—OH) (CH₂—CH₂—OH)
—CONH—C(CH₂—OH)₃ and
—CONH—CH(CH₂—OH)(CHOH—CH₂—OH).

More preferably the R groups will be equal or different and denote one or more moieties of the formulas —CON(CH₃)CH₂—CHOH—CH₂OH, —CONH—CH₂—CHOH—CH₂—OH, —CONH—CH—(CH₂—OH)₂, —CON—(CH₂—CH₂—OH)₂ or —CONH—CH₂—CHOH—CH₂—OH, —NHCOCH₂OH and —N(COCH₂OH)— mono, bis or tris-hydroxy C₁₋₄ alkyl, and even more preferably all R groups are equal and denote one of these moieties. Most preferably all R groups are the same and are —CON(CH₃)CH₂—CHOH—CH₂OH or —CONH—CH₂—CHOH—CH₂—OH.

Thus, examples of preferred structures according to the invention include the compounds of formula (II):

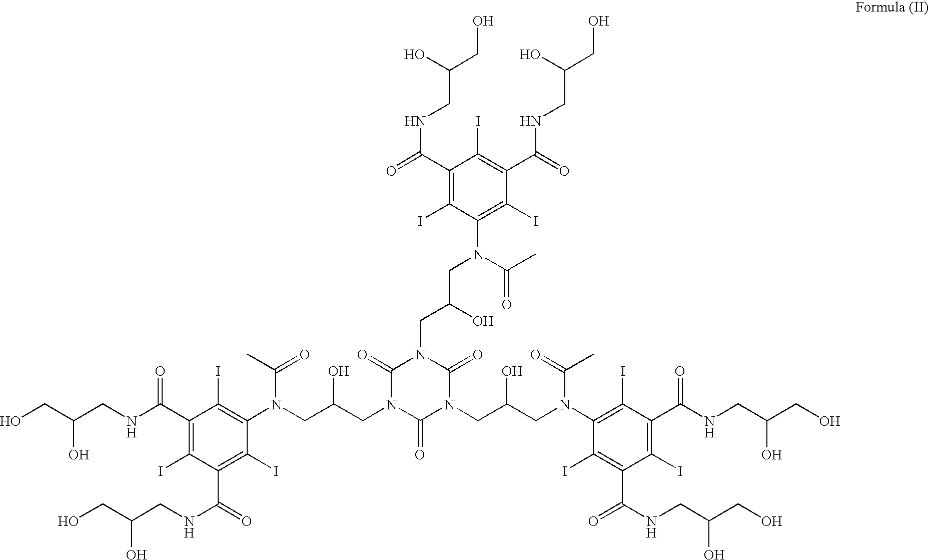

The compounds of formula (I) will attain a star-form with the relatively bulky iodinated phenyl substituents filling up the area between the 3 arms of the star. The molecule will therefore adopt a relatively round or globular form. Globular molecules will usually have enhanced solubility compared with similar molecules with a more planar structure.

The scaffolding heterocyclic cyanuric acid will itself contribute to the solubility of the compound of formula (I) by presenting its polar carboxylic groups to the solvent.

At an iodine concentration of 320 mg/ml which is a common concentration for commercially available iodinated contrast media, the concentration of the compound of formula (I) will be approximately 0.28 M (Molar). The contrast medium will also be hypoosmolar at this iodine concentration, and this is an advantageous property with regards to the nephrotoxicity of the contrast medium. It is also possible to add electrolytes to the contrast medium to lower the cardiovascular effects as explained in WO 90/01194 and WO 91/13636.

Compounds of formula (I) also comprises optical active isomers. Both enantiomerically pure products as well as mixtures of optical isomers are included.

The compounds of the invention may be used as contrast agents and may be formulated with conventional carriers and excipients to produce diagnostic contrast media.

Thus viewed from a further aspect the invention provides a diagnostic agent, and in particular a X-ray diagnostic agent comprising a compound of formula (I) as described above.

Further, the invention provides a diagnostic composition comprising a diagnostic agent, and in particular a X-ray diagnostic agent, of a compound of formula (I) as described above together with at least one physiologically tolerable carrier or excipient, e.g. in aqueous solution for injection, optionally together with added plasma ions or dissolved oxygen.

Still further, the invention provides methods of diagnosis and methods of imaging, specifically X-ray imaging, which comprises the administration of compounds of formula (I) to the human or animal body, examining the body with a diagnostic device and compiling data from the examination and optionally reaching to a diagnosis.

The diagnostic composition of the invention may be in a ready to use form of a suitable concentration or may be a concentrate form for dilution prior to administration. Generally compositions in a ready to use form will have iodine concentrations of at least 100 mg l/ml, preferably at least 150 mg l/ml, with concentrations of at least 300 mg l/ml, e.g. 320 mg l/ml being preferred. The higher the iodine concentration, the higher is the diagnostic value in the form of X-ray attenuation of the contrast media. However, the higher the iodine concentration the higher is the viscosity and the osmolality of the composition. Normally the maximum iodine concentration for a given contrast media will be determined by the solubility of the contrast enhancing agent, e.g. the iodinated compound, and the tolerable limits for viscosity and osmolality.

For contrast agent compositions or media which are administered by injection or infusion, the desired upper limit for the solution's viscosity at ambient temperature (20° C.) is about 30 mPas, however viscosities of up to 50 to 60 mPas and even more than 60 mPas can be tolerated. For contrast media given by bolus injection, e.g. in angiographic procedures, osmotoxic effects must be considered and preferably the osmolality should be below 1 Osm/kg H₂O, preferably below 850 mOsm/kg H₂O and more preferably about 300 mOsm/kg H₂O.

With the compounds of the invention such viscosity, osmolality and iodine concentrations targets can be met. Indeed, effective iodine concentrations can be reached with hypotonic solutions. It may thus be desirable to make up the solution's tonicity by the addition of plasma cations so as to reduce the toxicity contribution that derives from the imbalance effects following bolus injection. Such cations will desirably be included in the ranges suggested in WO 90/01194 and WO 91/13636.

In particular, addition of sodium and calcium ions to provide a contrast medium isotonic with blood for all iodine concentrations is desirable and obtainable. The plasma cations may be provided in the form of salts with physiologically tolerable counterions, e.g. chloride, sulphate, phosphate, hydrogen carbonate etc., with plasma anions preferably being used.

The compounds of formula (I) are produced in an one-pot synthesis where tris-(2,3-epoxypropyl)isocyanurate is reacted with a N-acylated triiodophenyl group substituted by R¹ and R² or R³ and R⁴ or R⁵ and R⁶ as illustrated in the example below.

The acylamino-triiodophenyl derivate is dissolved in aqueous methanol in presence of alkali hydroxide. Optionally the start pH can be adjusted with boric acid before addition of the alkylating isocyanurate derivative. Solvolysis of the isocyanurate ring can be suppressed by suitable low the reaction temperature and corresponding short reaction time. The reaction is stopped by neutralisation to pH<7 and the product is isolated by preparative HPLC.

The invention is further illustrated with the non-limiting example below:

### EXAMPLE

### 1,3,5-tris-(3-{acetyl-[3,5-bis-(2,3-dihydroxy-propyl-carbamoyl)-2,4,6-triiodo-phenyl]-amino}-2-hydroxy-propyl)-[1,3,5]triazinane-2,4,6-trione

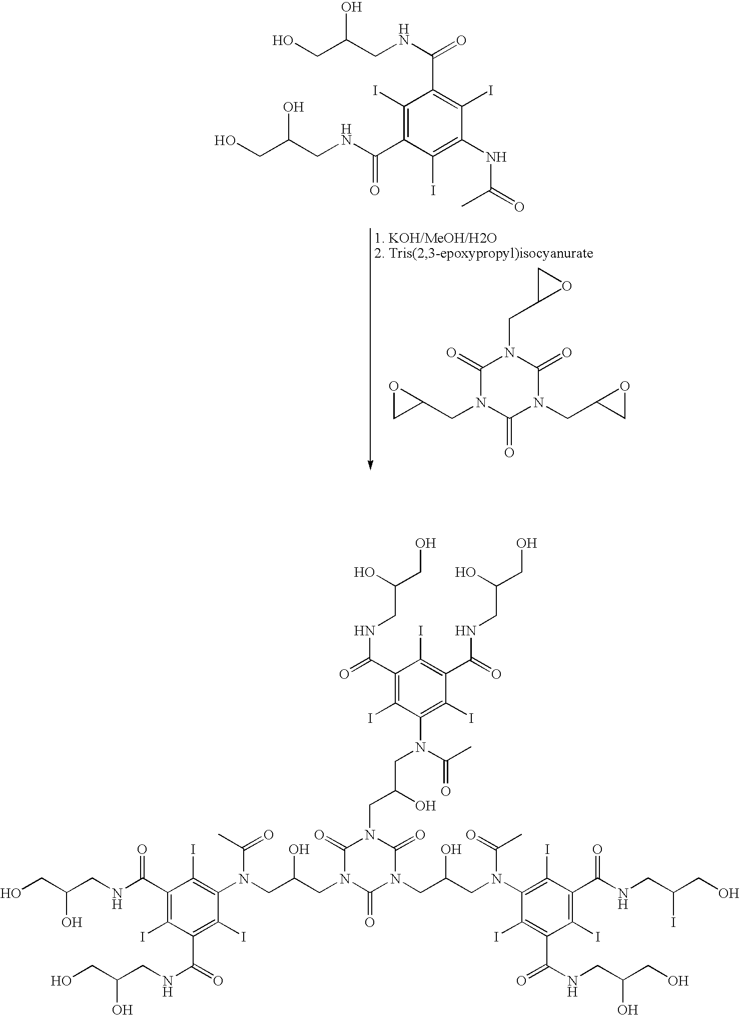

At 23-50° C. 5-Acetylamino-N,N′-bis-(2,3-dihydroxy-propyl)-2,4,6-triiodo-isophthalamide (400 g, 535.4 mmol) was dissolved in a solution of KOH (35.8 g, 638.7 mmol) in water (238 ml) and methanol (238 ml). To the clear solution boric acid (21.55 g, 348.5 mmol) is added and stirred for 0.5-3 h. At 10° C. Tris(2,3-epoxypropyl) isocyanurate (26.85 g, 90.3 mmol) was added and stirred for 19 h. The reaction was stopped by adding water (900 ml) and neutralization with 18.4% aqueous HCl to pH 4-5. A white precipitate (starting material) was filtered and the filter-cake washed with water. Salts were removed from the filtrate by ion exchangers Amberlite 200C and IRA67. The solution contains 68 HPLC area % target compound (ca. 150 g) and the product was isolated by prep. HPLC.

HPLC/MS (TOF ES+, m/e): 2538.8 [M⁺], 1269.8 [M²⁺].

¹H NMR (d6-DMSO): 8.1-8.6 (m, 6H, NH), 4.4-5.0 (m, 15H, OH), 4.2-4.4 (m, 3H, CH₂—CH—CH₂), 4.1-2.8 (m, 42H), 2.1 & 1.8 (m, 9H, CO—CH₃).

IR: 3276 (m), 1638 (s), 1560 (m), 1460 (s), 1261 (m), 1109 (m), 1039 (m), 765 (m).

1. Compounds of formula (I) 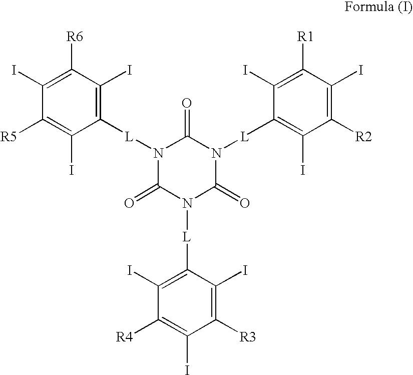
wherein:
each L are the same or different and denote a linker of formula —N(Ac)—CH₂—CHOH—CH₂— wherein Ac denotes a C₁ to C₅ organic acid residue;
each of the substituents R¹, R², R³, R⁴, R⁵ and R⁶ may be the same or different and denote a hydrogen atom or a non-ionic hydrophilic moiety, provided that at least one R group is a hydrophilic moiety
or salts or optical active isomers thereof.

2. Compounds as claimed in claim 1 wherein each of R¹, R², R³, R⁴, R⁵ and R⁶ may be the same or different and are a straight chain or branched chain C₁₋₁₀ alkyl groups optionally with one or more CH₂ or CH moieties replaced by oxygen or nitrogen atoms and optionally substituted by one or more groups selected from oxo, hydroxyl, amino or carboxyl derivative, and oxo substituted sulphur and phosphorus atoms.

3. Compounds as claimed in claim 1 wherein each of R¹, R², R³, R⁴, R⁵ and R⁶ may be the same or different and are a straight chain or branched chain C₁₋₅ alkyl groups optionally with one or more CH₂ or CH moieties replaced by oxygen or nitrogen atoms and optionally substituted by one or more groups selected from oxo, hydroxyl, amino or carboxyl derivative, and oxo substituted sulphur and phosphorus atoms.

4. Compounds as claimed in claim 3 wherein each of R¹, R², R³, R⁴, R⁵ and R⁶ are hydrophilic moieties containing 1 to 6 hydroxy groups.

5. Compounds as claimed in claim 1 wherein each of R¹, R², R³, R⁴, R⁵ and R⁶ may be the same or different and are polyhydroxyalkyl, hydroxyalkoxyalkyl and hydroxypolyalkoxyalky and such groups attached to the phenyl group via an amide linkage such as hydroxyalkylaminocarbonyl, N-alkyl-hydroxyalkylaminocarbonyl and bis-hydroxyalkylaminocarbonyl groups.

6. Compounds as claimed in claim 1 wherein each of R¹, R², R³, R⁴, R⁵ and R⁶ are the same or different and are selected from groups of the formulas
—CONH—CH₂—CH₂—OH
—CONH—CH₂—CHOH—CH₂—OH
—CON(CH₃)CH₂—CHOH—CH₂OH
—CONH—CH—(CH₂—OH)₂
—CON—(CH₂—CH₂—OH)₂
—CONH₂
—CONHCH₃
—NHCOCH₂OH
—N(COCH₃)H
—N(COCH₃)C₁₋₃ alkyl
—N(COCH₃)-mono, bis or tris-hydroxy C₁₋₄ alkyl
—N(COCH₂OH)-hydrogen, mono, bis or tris-hydroxy C₁₋₄ alkyl
—N(CO—CHOH—CH₂OH)-hydrogen, mono, bis or trihydroxylated C₁₋₄ alkyl.
N(CO—CHOH—CHOH—CH₂OH)-hydrogen, mono, bis or trihydroxylated C₁₋₄ alkyl.
—N(COCH₂OH)₂
—CON(CH₂—CHOH—CH₂—OH) (CH₂—CH₂—OH)
—CONH—C(CH₂—OH)₃ and
—CONH—CH(CH₂—OH)(CHOH—CH₂—OH).

7. Compounds as claimed in claim 6 wherein each of R¹, R², R³, R⁴, R⁵ and R⁶ are the same or different and are selected from groups of the formulas —CON(CH₃)CH₂—CHOH—CH₂OH, —CONH—CH₂—CHOH—CH₂—OH, —CONH—CH—(CH₂—OH)₂, —CON—(CH₂—CH₂—OH)₂ or —CONH—CH₂—CHOH—CH₂—OH, —NHCOCH₂OH and —N(COCH₂OH)— mono, bis or tris-hydroxy C₁₋₄ alkyl.

8. Compounds as claimed in claim 7 wherein each of R¹, R², R³, R⁴, R⁵ and R⁶ are equal and are —CON(CH₃)CH₂—CHOH—CH₂OH or —CONH—CH₂—CHOH—CH₂—OH.

9. Compounds as claimed in claim 1 wherein Ac denotes a C₁ to C₅ organic acid residue such as the formyl, acetyl, propionyl, butyryl, isobutyryl and valeriyl.

10. Compounds as claimed in claim 1 wherein Ac denotes a hydroxylated or metoxylated C₁ to C₅ organic acid residue

11. Compounds as claimed in claim 1 wherein Ac denotes acetyl, hydroxyacetyl and metoxyacetyl moieties.

12. Compounds as claimed in claim 1 wherein the compounds claimed are 1,3,5-tris-(3-{acetyl-[3,5-bis-(2,3-dihydroxy-propyl-carbamoyl)-2,4,6-triiodo-phenyl]-amino}-2-hydroxy-propyl)-[1,3,5]triazinane-2,4,6-trione.

13. A diagnostic agent comprising compounds of formula (I) as defined in claim 1.

14. A diagnostic composition comprising compounds of formula (I) as defined in claim 1 together with a pharmaceutically acceptable carrier or excipient.

15. An X-ray diagnostic composition comprising compounds of formula (I) as defined in claim 1 together with a pharmaceutically acceptable carrier or excipient.

16. A method of diagnosis for the human or animal body comprising administration of compounds of formula (I) as defined in claim 1, examining the body with a diagnostic device and compiling data from the examination.

17. A method of imaging a human or animal body comprising administration of compounds of formula (I) as defined in claim 1, examining the body with a diagnostic device and compiling data from the examination and optionally analysing the data.

18. Compounds as claimed in claim 4 wherein the hydrophilic moieties contain 1 to 3 hydroxy groups.

19. A method of imaging a human or animal body as defined in claim 17 wherein the method of imaging is X-ray imaging.
